SEQUENCE LISTING (14 sequences)

SEQ ID NO: 1 - DNA, 51 bp, Homo sapiens
catggtggtcctctgcgtgggctccaaggggcaggtgtttgccacgtcggc

SEQ ID NO: 2 - DNA, 51 bp, Homo sapiens
catggtggtcctctgcgtgggctccgaggggcaggtgtttgccacgtcggc

SEQ ID NO: 3 - DNA, 28 bp, Homo sapiens
tggatgccctccagcgccagctcaacag

SEQ ID NO: 4 - DNA, 28 bp, Homo sapiens
tggatgccctccagctccagctcaacag

SEQ ID NO: 5 - DNA, 20 bp, Homo sapiens
cccttccccagacgagagca

SEQ ID NO: 6 - DNA, 20 bp, Homo sapiens
ctccacccatcccagcacat

SEQ ID NO: 7 - DNA, 30 bp, Homo sapiens
gcggcgacgccccttccccagacgagagca

SEQ ID NO: 8 - DNA, 26 bp, Homo sapiens
gcctgcctccacccatcccagcacat

SEQ ID NO: 9 - DNA, 25 bp, Homo sapiens
gccgacgtggcaaacacctgcccct

SEQ ID NO: 10 - DNA, 51 bp, Homo sapiens
catggtggtcctctgcgtgggctccagggggcaggtgtttgccacgtcggc

SEQ ID NO: 11 - DNA, 42 bp, Homo sapiens
tgtaaaacgacggccagttaccccgctcacccagctctgctc

SEQ ID NO: 12 - DNA, 42 bp, Homo sapiens
caggaaacagctatgacccccccaccccacttgcattccttc

SEQ ID NO: 13 - DNA, 25 bp, Homo sapiens
gagagcaggctggatgccctccagc

SEQ ID NO: 14 - DNA, 29 bp, Homo sapiens
tggatgccctccagcgtccagctcaacag

(19) United States
(12) Patent Application Publication
(10) Pub. No.: US 2005/0059032 A1
(43) Pub. Date: Mar. 17, 2005

(54) Methods & compositions relating to drug-induced arrhythmia

(75) Inventors: Patrice M. Milos, Cranston, RI (US); Amy Mank-Seymour, Madison, CT (US); Jodi Lynn Richmond, Mystic, CT (US); Aidan C. Power, East Lyme, CT (US)

Correspondence Address: PFIZER INC., PATENT DEPARTMENT, MS8260-1611, EASTERN POINT ROAD, GROTON, CT, 06340, US

(73) Assignee: Pfizer Inc

(21) Appl. No.: 10/741,183

(22) Filed: Dec. 19, 2003

Related U.S. Application Data
(60) Provisional application No. 60/435,470, filed on Dec. 21, 2002.

(51) Int. Cl. C12Q 1/68

(52) U.S. Cl. 435/6

(57) ABSTRACT

The present invention features polymorphisms in genes encoding cardiac potassium channels that are predictive of a subject's susceptibility to developing a drug-induced arrhythmia. The present invention also provides methods of genotyping subjects to determine whether they carry the polymorphisms.

### CROSS REFERENCE TO RELATED APPLICATION

This application claims priority of U.S. Provisional Application No. 60/435,470 filed Dec. 21, 2002.

### FIELD OF THE INVENTION

The present invention relates to technologies for assessing a subject's susceptibility to development of a drug-induced arrhythmia. The invention further relates to methods for screening candidate therapeutics that will be optimally effective for treating patients suffering from an arrhythmia. The invention also relates to methods for stratifying a patient in a clinical trial based on the presence or absence of a polymorphism in either the KCNH2 gene or the KCNQ1 gene. The invention further provides methods, compositions and kits useful for determining a patient's KCNH2 and/or KCNQ1 genotype.

### BACKGROUND OF THE INVENTION

Cardiac arrhythmias are a common cause of morbidity and mortality accounting for approximately 11% of all natural deaths (Kannel, et al., (1987) Am. Heart J., 113:799-804, Willich et al., (1987) Am. J. Cardiol., 60:801-806). Arrhythmias result from a deviation from the normal conduction process of the heart, where the coordinated and steady contraction of the atria and ventricles is interrupted. The resultant disruption of the coordinated contraction may result in a decrease in blood pressure and a concomitant loss in consciousness known as syncope. Prolonged disturbances in heart rhythm may further result in permanent brain damage and death.

Anti-arrhythmic medications are administered to prevent dangerous changes in heart rhythm. However, in certain instances, medications intended to alleviate a pre-existing arrhythmia can have the unintended consequence of causing an additional arrhythmia as a side effect in a subset of the patients that receive them. In fact, nearly every known antiarrhythmic has the potential to induce malignant arrhythmias (proarrhythmia) as an unintended side effect. Additionally, certain non-cardiac medications are known to interfere with the molecular mechanisms of cardiac contraction and can cause an arrhythmia. Arrhythmias induced by medication are termed drug-induced arrhythmias to differentiate them from arrhythmias with other etiologies. The increased susceptibility to arrhythmia following the intake of a drug is currently difficult to treat, as the predisposing factors that lead to drug-induced arrhythmia have not previously been identified.

Certain class III and IA anti-arrhythmic medications function by prolonging the cardiac action potential by blocking the rapidly activating cardiac potassium current I_{Kr}. Antiarrhythmics are traditionally classified into four groups based on their molecular targets of action. Class I antiarrhythmics bind to cardiac sodium (Na) channels, while Class II drugs act by blocking the beta adrenergic receptor; Class III antiarrhythmics bind potassium (K) channels, while Class IV drugs block calcium channels. Furthermore, Class I antiarrhythmics have been traditionally sub-classified as follows: Class Ia drugs decrease the maximal velocity of the cardiac action potential (Vmax) and increase the refractory period of the conduction tissue, while Class Ib drugs decrease the duration of that same action potential, and Class Ic agents depress Vmax with no concomitant effect on the refractory period.

As stated earlier, nearly every known antiarrhythmic in use today has the potential to induce malignant arrhythmias themselves (proarrhythmia), leading to a narrow therapeutic window. More specifically, Class Ia and III medications have a particular proarrhythmic side effect of inducing a malignant arrhythmia termed Torsade de Pointes (TdP), or polymorphic ventricular tachycardia (PMVT). It is thought that this arrhythmia occurs when there is so-called dispersion of the repolarization and refractory periods of the ventricular tissue. That is, there is heterogeneity in the ability of ventricular tissue to respond to the normal depolarization wave that results from normal conduction. As a result, aberrant conduction ensues, leading to the inability of the ventricles to perform their primary function, pump blood to the rest of the body and support a normal blood pressure. This particular aberrant ventricular conduction is manifested on an electrocardiogram as an undulating sinusoidal pattern, usually at a rate much faster than the normal sinus rhythm, hence the terms Torsade de Pointes and polymorphic ventricular tachycardia. If this type of arrhythmia persists, blood pressure decreases significantly, leading to underperfusion of blood to the brain, and loss of consciousness (syncope). Continued persistence of such arrhythmias results in death. The initiation of TdP is unpredictable and occurs as a paroxysmal event. Once recognized, TdP can be easily treated with several modalities, including electrical cardioversion, magnesium infusion, or antitachyardia (overdrive) pacing with an artificial pacemaker. However, if an episode of TdP occurs outside of medical observation, the results can be fatal, as time to treatment is crucial.

TdP and PMVT may be induced by medications de novo, as outlined above. However, in many instances abnormalities in the normal sinus rhythm ECG can be detected that increase the likelihood that such a patient will be susceptible to drug-induced TdP or PMVT. In particular, the QT interval, which reflects the repolarization of ventricular myocardium, may be abnormally prolonged by certain medications, including Class Ia and Class III antiarrhythmics, tricyclic antidepressants, certain antihistamines, and several other medications. This prolonged QT, or long QT, reflects the above-mentioned dispersion or heterogeneity of ventricular repolarization that likely leads to a state susceptible to TdP or PMVT.

Although the occurrence of TdP and other malignant arrhythmias has dire consequences, the incidence in the general population and even in patients taking antiarrhythmics is low. That's why these medications have been approved for use, and are currently available. However, it may be possible that a certain subset of individuals may be predisposed to a higher likelihood of developing TdP or other drug-induced arrhythmias based on their genetic makeup. In particular, since these above-described medications are thought to not only have their therapeutic effect by interacting with ion channels, but to also have their proarrhythmic effects through interactions with ion channels, it seems plausible that polymorphisms in these ion channels may account for these differences in susceptibility to the proarrhythmic effects of these drugs.

If a screening genetic examination were available to predict those patients susceptible to these proarrhythmic effects, a significant benefit would be possible. Currently, not only are these proarrhythmic effects difficult to predict, but as a result, patients administered certain antiarrhythmics (dofetilide, ibutilide, sotalol, etc) are required to be monitored closely in an inpatient setting during initiation of these medications. Despite this close monitoring, a subset of these patients who tolerate the drug initially can still develop TdP paroxysmally while at home. Therefore, it is clear that the ability to screen for those patients likely to develop a drug-induced arrhythmia has significant importance for the medical community.

### SUMMARY OF THE INVENTION

The present invention is directed to single nucleotide polymorphisms (SNPs) that are important in the molecular and genetic pathogenesis of drug-induced arrhythmia and to methods of predicting and/or diagnosing a subject's susceptibility to drug-induced arrhythmia.

According to the present invention, susceptibility to drug-induced arrhythmia is determined by analyzing the DNA sequence of either the KCNH2 or KCNQ1 gene of a patient for the presence of a polymorphism. The present invention is further directed to methods of screening for the presence of KCNQ1 or KCNH2 gene variants, which increase the probability of the patient developing drug-induced arrhythmia. Early detection of a subject's susceptibility to a drug-induced arrhythmia will permit a more informed choice of medical regimen and alleviate the need to hospitalize patients receiving medications which may have the unintended consequence of increasing the patient's susceptibility to TdP. Specifically, patients receiving anti-arrhythmic or other medications which prolong the QT interval and, therefore, increase the likelihood of TdP are screened for polymorphisms in the KCNH2 channel gene and in the KCNQ channel gene. The detection of, for example, a R1047L mutation in the KCNH2 channel or a K218E mutation in the KCNQ1 gene is indicative of an increased likelihood of developing TdP following the administration of a medication that prolongs the QT interval, such as a class III or class Ia anti-arrhythmic. The disclosed mutations occur in much higher frequencies in patients that developed TdP following treatment with dofetilide, a class III anti-arrhythmic, then in patients undergoing dofetilide treatment without any complications.

According to the diagnostic and prognostic methods of the present invention, alteration of the wild type KCNQ1 or KCNH2 gene is detected and deemed indicative of the subject's susceptibility to drug-induced arrhythmia. In addition, the method can be performed by detecting the wild type KCNQ1 or KCNH2, and thus confirming the lack of a susceptibility to drug-induced arrhythmia as a result of this locus.

Other features and advantages will be apparent based on the following DETAILED DESCRIPTION OF THE INVENTION and the appendant claims.

### BRIEF DESCRIPTION OF THE FIGURES

FIG. 1. Exon 3 Polymorphism: KCNQ1 Alleles

FIG. 2. Exon 13 Polymorphism: KCNH2 Alleles

### DETAILED DESCRIPTION OF THE INVENTION

Definitions

For convenience, the meaning of certain terms and phrases employed in the specification, examples, and appended claims are provided below.

The term “allele” as used herein refers to one or more alternative forms of a gene, located at the same locus in a set of homologous chromosomes. For example, the KCNH2 gene has multiple different alleles, two of which are described in this application.

The term “amplification” as used herein with reference to nucleic acids is intended to encompass any method of generating one or more copies of a nucleic acid, either in single or double stranded form. Such methods include but are not limited to polymerase chain reaction (PCR) and replication of the nucleic acid in cells.

The term “acquired long QT syndrome” as used herein refers to any variation from the normal sinus rhythm of the heart, where the variation is associated with a prolonged QT interval, and the variation is induced by the administration of medication. The medication may either be of any sort, including anti-arrhythmic medications, other cardiac medications, and even non-cardiac medications.

The term “arrhythmia” as used herein refers to any variation from the normal sinus rhythm of the heart, where the variation is most often caused by a disturbance in the conduction properties of the atria and/or ventricles. Such variations include, but are not limited to, tachy-arrhythmia, atrial fibrilation, atrial flutter, atrial tachycardia, supraventricular tachycardia, AV nodal re-entry tachycardia, or ventricular tachycardia.

The term “biological sample” as used herein refers to any cell, fluid or tissue that is collected from a subject in order to obtain nucleic acid samples for use in the methods described herein. Most simply, blood can be drawn from the subject and DNA may be extracted from the blood sample. Alternatively, a buccal swab, a hair follicle preparation, spinal tap, tissue smear or a nasal aspirate may be used to provide the requisite nucleic acid sample.

The term “drug-induced arrhythmia” as used herein refers to any variation from the normal sinus rhythm of the heart associated with the administration of a medication. In certain embodiments the variation may be caused by a disturbance in the conduction properties of the atria and/or ventricles. The medication may either be of any sort, including anti-arrhythmic medications, other cardiac medications, and even non-cardiac medications.

“Increased risk” as used herein refers to a higher frequency of occurrence of a disease or disorder in a population in comparison to the frequency of occurrence of the disease or disorder in a control population. A factor identified to be associated with increased risk is termed a “risk factor.” In an exemplary embodiment, a particular polymorphic allele may be a risk factor for drug-induced arrhythmia.

The term “K218E polymorphism” as used herein refers to a base pair variation in the nucleic acid sequence of exon 3 of KCNQ1 that results in a transition from K to E at position 218 of the amino acid sequence of KCNQ1 and alleles in linkage disequilibrium therewith.

The term “long QT interval” as used herein refers to a QT interval, the time interval between the Q wave and the T wave of the electrocardiogram, longer then approximately one-half of the R to R interval.

As used herein, the term “nucleic acid” refers to polynucleotides or oligonucleotides such as deoxyribonucleic acid (DNA), and, where appropriate, ribonucleic acid (RNA). The term should also be understood to include, as equivalents, analogs of either RNA or DNA made from nucleotide analogs (e.g., peptide nucleic acids) and as applicable to the embodiment being described, single (sense or antisense) and double-stranded polynucleotides.

The term “polymorphism” as used herein refers to variations in a DNA sequence among individuals. A portion of a gene having at least two different forms, e.g., two different nucleotide sequences, is referred to as a “polymorphic region of a gene.” For example, a polymorphic region may involve single or multiple base pair alterations including, without limitation, insertions, deletions, substitutions, and/or sequence repeats.

The term “R1047L polymorphism” as used herein refers to a base pair variation in the nucleic acid sequence of exon 13 of KCNH2 that results in a transition from R to L at position 1047 of the amino acid sequence of KCNH2 and alleles in linkage disequilibrium therewith.

As used herein, the term “specifically hybridizes” or “specifically detects” refers to the ability of a nucleic acid molecule to hybridize to at least approximately 6 consecutive nucleotides of a sample nucleic acid.

The term “treating” as used herein is intended to encompass curing as well as ameliorating at least one symptom of a disease or at least one abnormality associated with a disorder. Treating a drug-induced arrhythmia may be carried out by administering a therapeutic that alleviates the arrhythmia. Alternatively, treating a drug-induced arrhythmia may be carried out by administering a therapeutic that modifies the risk factors associated with the arrhythmia.

The terms “wild-type allele” or “normal allele” refer to an allele of a gene which, when present in two copies in a subject, results in a wild-type phenotype. There can be several different wild-type alleles of a specific gene, since certain nucleotide changes in a gene may not affect the phenotype of a subject having two copies of the gene with the nucleotide changes. In general a “wild-type allele” is the most common allele in a population.

Therapeutics that Lead to Drug-induced Arrhythmias

Drug-induced, or acquired, arrhythmias may be triggered either by anti-arrhythmic medications, other cardiac medications, or by non-cardiac medications. Anti-arrhythmic medications known to trigger drug-induced arrhythmia include class III antiarrhythmics including, but not limited to, dofetilide or solatol, and class la antiarrhythmics including, but not limited to, procainamide.

Drug-induced cardiac arrhythmias also occur during treatment with non-cardiac drugs that have unintended effects on cardiac repolarization. Examples of drugs associated with drug-induced arrhythmias include, but are not limited to: quinidine, terfenadine, cisapride, erythromycin, amiodarone, phenothiazines, tricyclic antidepressants, and some diuretics (see e.g., Roden, D. M., (1998) Am J Cardiol, 82(4A):491-571). Although the molecular mechanisms of drug-induced arrhythmia are not yet known, it is of interest that many of these medications block KCNH2 channels (for a review see Keating, M. T., et al., (2001) Cell, 104:569-580). Additionally, as many as 10% of patients treated with quinidine, sotalol, and ibutilide will develop excessive QT interval prolongation or exhibit the precipitous occurrence of torsade de pointes. This unpredictable adverse reaction can occur in the absence of identifiable risks factors such as hypokalemia, hypomagnesemia, concomitant treatment with other I_{Kr} blockers, and recent conversion from atrial fibrillation. See Tan, H. L., et al., Ann. Intern. Med. 122:701-714 (1995).

The ability to rapidly genotype patients promises to radically change the testing and development of therapeutic or disease-preventative substances. Currently, the effectiveness of a substance for treating or preventing a disease is assessed by testing it on a pool of patients. Many variables in the patient pool are controlled for, but the effects of genetic variability are not typically assessed. A drug may be statistically ineffective when examined in a diverse pool of patients and yet be highly effective for a select group of patients with particular genetic characteristics. Similarly, a drug may cause unwanted side effects only in a genetically defined subpopulation of patients, but not in the patient pool as a whole. Unless patients are separated by genotype, many drugs with great promise for selected populations are likely to be rejected as useless for the population as a whole. Additionally, the identification of polymorphisms which underlie a patient's susceptibility to dangerous drug side effects will allow physicians to screen for the presence of these predisposing factors prior to administration of the medication. This ability to screen a patient a priori would minimize the likelihood of the patient developing the side effect in the course of treatment.

If a patient pool can be segregated into groups based on genotype, drugs can be re-tested for their ability to affect genetically defined subgroups of patients. This type of screening may allow the resurrection of failed compounds, the identification of new or more refined compounds, the identification of new uses for well-known compounds, and the identification of a population who will not develop potentially dangerous side effects upon the administration of the therapeutic.

Polymorphisms Associated with Increased Susceptibility to Drug-induced Arrhythmia

The present invention is based, at least in part, on the identification of novel alleles of the KCNH2 gene and KCNQ1 gene, which are useful for identifying patients with an increased susceptibility to drug-induced arrhythmia. Therefore, detection of these alleles in a subject indicates that the subject has an altered susceptibility to the development (increased or decreased) of a side effect induced by medication. Further, detection of the alleles aids in determining the choice of therapeutic which will be optimally useful for patients with a particular KCNH2 or KCNQ1 genotype.

The mutations in the KCNH2 gene and in the KCNQ1 gene disclosed herein may be used as predictive tools in measuring a patient's susceptibility to TdP. Specifically, two independent mutations were determined to occur with a higher frequency in patients who developed TdP following dofetilide treatment. In the KCNH2 potassium channel, the polymorphism results in an amino acid alteration at position 1047 in the sequence. Specifically, an arginine is replaced with a leucine. In the KCNQ1 potassium channel, the polymorphism results in an amino acid alteration at position 218 in the sequence. Specifically, a lysine is replaced with a glutamate. In light of these discoveries, the analysis of a subject's genotype for the presence of either of the above described polymorphisms would permit the subject to be screened for the risk of developing TdP prior to the administration of any medication.

In a merely illustrative embodiment, the method includes the steps of (i) collecting a sample of cells from a patient, (ii) isolating nucleic acid (e.g., genomic, mRNA or both) from the cells of the sample, (iii) contacting the nucleic acid sample with one or more primers which specifically hybridize 5′ and 3′ to at least one allele of either the KCNQ1 or KCNH2 under conditions such that hybridization and amplification of the allele occurs, and (iv) detecting the amplification product. These detection schemes are especially useful for the detection of nucleic acid molecules if such molecules are present in very low numbers.

In addition to the alleles described herein, one of skill in the art, based on the teachings herein can readily identify other alleles (including polymorphisms and mutations) that are associated with drug-induced arrhythmia or alleles that are in linkage disequilibrium with a drug-induced arrhythmia associated allele. For example, a nucleic acid sample from a first group of subjects without drug-induced arrhythmia can be collected, as well as DNA from a second group of subjects with a drug-induced arrhythmia. The nucleic acid sample can then be compared to identify those alleles that are over-represented in the second group as compared with the first group, wherein such alleles are presumably associated with a drug-induced arrhythmia. Alternatively, alleles that are in linkage disequilibrium with a drug-induced arrhythmia associated allele can be identified, for example, by genotyping a large population and performing statistical analysis to determine which alleles appear more commonly together than expected. Preferably the group is chosen to be comprised of genetically related individuals. Genetically related individuals include individuals from the same race, the same ethnic group, or even the same family. As the degree of genetic relatedness between a control group and a test group increases, so does the predictive value of polymorphic alleles which are ever more distantly linked to a disease-causing allele. This is because less evolutionary time has passed to allow polymorphisms which are linked along a chromosome in a founder population to redistribute through genetic cross-over events. Thus, race-specific, ethnic-specific, and even family-specific diagnostic genotyping assays can be developed to allow for the detection of disease alleles which arose at ever more recent times in human evolution, e.g., after divergence of the major human races, after the separation of human populations into distinct ethnic groups, and even within the recent history of a particular family line.

Linkage disequilibrium between two polymorphic markers or between one polymorphic marker and a disease-causing mutation is a meta-stable state. Absent selective pressure or the sporadic linked reoccurrence of the underlying mutational events, the polymorphisms will eventually become disassociated by chromosomal recombination events and will thereby reach linkage equilibrium through the course of human evolution. Thus, the likelihood of finding a polymorphic allele in linkage disequilibrium with a disease or condition may increase with changes in at least two factors: decreasing physical distance between the polymorphic marker and the disease-causing mutation, and decreasing number of meiotic generations available for the dissociation of the linked pair. Consideration of the latter factor suggests that, the more closely related two individuals are, the more likely they will share a common parental chromosome or chromosomal region containing the linked polymorphisms and the less likely that this linked pair will have become unlinked through meiotic cross-over events occurring with each generation. As a result, the more closely related two individuals are, the more likely it is that widely spaced polymorphisms may be co-inherited. Thus, for individuals related by common race, ethnicity or family, the reliability of ever more distantly spaced polymorphic loci can be relied upon as an indicator of inheritance of a linked disease-causing mutation.

Genetic screening (also called genotyping or molecular screening), can be broadly defined as testing to determine if a patient has mutations (or alleles or polymorphisms) that either cause a disease state, contribute to a disease state (i.e., are a risk factor associated with a disease state), are “linked” to the mutation causing a disease state, or are “linked” to the mutation which contributes to the disease state. Linkage refers to the phenomenon wherein DNA sequences that are close together in the genome have a tendency to be inherited together. Two sequences may be linked because of some selective advantage of co-inheritance. More typically, however, two polymorphic sequences are co-inherited because of the relative infrequency with which meiotic recombination events occur within the region between the two polymorphisms. The co-inherited polymorphic alleles are said to be in linkage disequilibrium with one another because, in a given human population, they tend to either both occur together or else not occur at all in any particular member of the population. Indeed, where multiple polymorphisms in a given chromosomal region are found to be in linkage disequilibrium with one another, they define a quasi-stable genetic “haplotype.” In contrast, recombination events occurring between two polymorphic loci cause them to become separated onto distinct homologous chromosomes. If meiotic recombination between two physically linked polymorphisms occurs frequently enough, the two polymorphisms will appear to segregate independently and are said to be in linkage equilibrium.

While the frequency of meiotic recombination between two markers is generally proportional to the physical distance between them on the chromosome, the occurrence of “hot spots” as well as regions of repressed chromosomal recombination can result in discrepancies between the physical and recombination distance between two markers. Thus, in certain chromosomal regions, multiple polymorphic loci spanning a broad chromosomal domain may be in linkage disequilibrium with one another, and thereby define a broad-spanning genetic haplotype. Furthermore, where a disease-causing mutation is found within or in linkage with this haplotype, one or more polymorphic alleles of the haplotype can be used as a diagnostic or prognostic indicator of the likelihood of developing the disease. This association between otherwise benign polymorphisms and a disease-causing polymorphism occurs if the disease mutation arose in the recent past, so that sufficient time has not elapsed for equilibrium to be achieved through recombination events. Therefore identification of a human haplotype that spans or is linked to a disease-causing mutational change, serves as a predictive measure of an individual's likelihood of having inherited that disease-causing mutation. Importantly, such prognostic or diagnostic procedures can be utilized without necessitating the identification and isolation of the actual disease-causing lesion. This is significant because the precise determination of the molecular defect involved in a disease process can be difficult and laborious, especially in the case of multifactorial diseases such as coronary artery disease.

Indeed, the statistical correlation between a disease state and a polymorphism does not necessarily indicate that the polymorphism directly causes the disorder. Rather the correlated polymorphism may be a benign allelic variant which is linked to (i.e., in linkage disequilibrium with) a disorder-causing mutation which has occurred in the recent human evolutionary past, so that sufficient time has not elapsed for equilibrium to be achieved through recombination events in the intervening chromosomal segment. Thus, for the purposes of diagnostic and prognostic assays for a particular disease, detection of a polymorphic allele associated with that disease can be utilized without consideration of whether the polymorphism is directly involved in the etiology of the disease. Furthermore, where a given benign polymorphic locus is in linkage disequilibrium with an apparent disease-causing polymorphic locus, still other polymorphic loci which are in linkage disequilibrium with the benign polymorphic locus are also likely to be in linkage disequilibrium with the disease-causing polymorphic locus. Thus, these other polymorphic loci will also be prognostic or diagnostic of the likelihood of having inherited the disease-causing polymorphic locus. Indeed, a broad-spanning human haplotype (describing the typical pattern of co-inheritance of alleles of a set of linked polymorphic markers) can be targeted for diagnostic purposes once an association has been drawn between a particular disease or condition and a corresponding human haplotype. Thus, the determination of an individual's likelihood for developing a particular disease or condition can be made by characterizing one or more disease-associated polymorphic alleles (or even one or more disease-associated haplotypes) without necessarily determining or characterizing the causative genetic variation.

Sample Collection

Any cell type or tissue may be utilized to obtain nucleic acid samples for use in the methods described herein. Most simply, blood can be drawn from the subject and DNA may be extracted from the blood sample. Alternatively, a buccal swab, a hair follicle preparation, spinal tap, tissue smear or a nasal aspirate is used as a source of cells to provide the requisite nucleic acid sample.

Tissue samples may be stored before analysis by well-known storage means that will preserve the nucleic acids for later analysis. Such methods include, but are not limited to, quick freezing, or a controlled freezing regime, in the presence of a cryoprotectant, for example, dimethyl sulfoxide (DMSO), glycerol, or propanediol-sucrose.

Diagnostic procedures may also be performed in situ directly upon tissue sections (fixed and/or frozen) of patient tissue obtained from biopsies or resections, such that no nucleic acid purification is necessary. Nucleic acid reagents may be used as probes and/or primers for such in situ procedures (see, for example, Nuovo, G. J., 1992, PCR in situ hybridization: protocols and applications, Raven Press, NY).

Amplification Techniques

The nucleic acids present in the above-described tissue samples may be amplified by any conventional means known in the art. In one embodiment, the nucleic acid sample may be amplified by polymerase chain reaction (PCR) methods. Alternatively, the amplification may be accomplished by in vitro cell culture methods.

The allelic discrimination techniques described below may also comprise the step of amplifying the nucleic acid before analysis. Amplification techniques are known to those of skill in the art and include, but are not limited to cloning, PCR, polymerase chain reaction of specific alleles (ASA), ligase chain reaction (LCR), nested polymerase chain reaction, self sustained sequence replication (Guatelli, J. C. et al., Proc. Natl. Acad. Sci. (USA) 87:1874-1878 (1990)), transcriptional amplification system (Kwok, D. Y. et al., Proc. Natl. Acad. Sci. (USA) 86:1173-1177 (1989)), and Q-Beta Replicase (Lizardi, P. M. et al., Bio/Technology 6:1197 (1988)).

Amplification products may be assayed in a variety of ways, including size analysis, restriction digestion followed by size analysis, detecting specific tagged oligonucleotide primers in the reaction products, allele-specific oligonucleotide (ASO) hybridization, allele specific 5′ exonuclease detection, sequencing, hybridization, and the like.

PCR based detection means can include multiplex amplification of a plurality of markers simultaneously. For example, it is well known in the art to select PCR primers to generate PCR products that do not overlap in size and can be analyzed simultaneously. Alternatively, it is possible to amplify different markers with primers that are differentially labeled and thus can each be differentially detected. Of course, hybridization based detection means allow the differential detection of multiple PCR products in a sample. Other techniques are known in the art to allow multiplex analyses of a plurality of markers.

Appropriate probes may be designed to hybridize to specific regions of the KCNQ1 or KCNH2 gene. Primers useful for amplifying regions of the KCNQ1 or KCNH2 gene will be apparent to one of skill in the art in light of the present disclosure. Reasonable primers include those which hybridize within about 1 kb of the designated primer, and which further are anywhere from about 17 bp to about 27 bp in length. A general guideline for designing primers for amplification of unique human chromosomal genomic sequences is that they possess a melting temperature of at least about 50° C., wherein an approximate melting temperature can be estimated using the formula Tₘₑₗₜ=[2×(# of A or T)+4×(# of G or C)]. These probes may incorporate other regions of the relevant genomic locus, including intergenic sequences. Indeed, the KCNQ1 or KCNH2 gene spans some 25,000 base pairs and, assuming an average of one single nucleotide polymorphism every 1,000 base pairs, includes some 25 SNP loci alone. Yet other polymorphisms available for use with the immediate invention are obtainable from various public sources. For example, the human genome database collects intragenic SNPs, is searchable by sequence and currently contains approximately 2,700 entries. Also available is a human polymorphism database maintained by the Massachusetts Institute of Technology or a database maintained by the SNP Consortium. From such sources, SNPs as well as other human polymorphisms may be found.

Accordingly, the nucleotide segments of the invention may be used for their ability to selectively form duplex molecules with complementary stretches of the KCNQ1 or KCNH2 gene region. The design of appropriate probes for this purpose requires consideration of a number of factors. For example, fragments having a length of between 10, 15, or 18 nucleotides to about 20, or to about 30 nucleotides, will find particular utility. Longer sequences, e.g., 40, 50, 80, 90, 100, even up to full length, are even more preferred for certain embodiments. Lengths of oligonucleotides of at least about 18 to 20 nucleotides are well accepted by those of skill in the art as sufficient to allow sufficiently specific hybridization so as to be useful as a molecular probe. Furthermore, depending on the application envisioned, one will desire to employ varying conditions of hybridization to achieve varying degrees of selectivity of probe towards target sequence. For applications requiring high selectivity, one will typically desire to employ relatively stringent conditions to form the hybrids. For example, relatively low salt and/or high temperature conditions, such as provided by 0.02 M-0.15M NaCl at temperatures of about 50° C. to about 70° C. Such selective conditions may tolerate little, if any, mismatch between the probe and the template or target strand.

Genotyping Methods

SNPs are useful genetic tools, particularly as disease markers. SNPs are generally viewed as the preferred genetic markers for disease susceptibility, over, e.g., microsatellites, because (a) SNPs are very prevalent in the genome, meaning that it is likely that a SNP can be identified in the locus of interest, (b) a subset of SNPs will directly affect the polypeptide product of the gene revealing novel molecular mechanisms of a disease, (c) they are more stably inherited then other genetic markers and, finally, (4) existing genotyping methods provide for relatively rapid and efficient means of screening a subject for the presence or absence of the SNP of interest (Landegren, et al., (1998) Genome Research, 8:769-776).

Multiple genotyping methods are currently available. While all of the currently available methods involve amplification of the target sequence, this invention also contemplates methods that do not require sequence amplification. A convenient summary of the currently available methods is disclosed in Table I of Landegren, et al., (1998) Genome Research, 8:769-776. Methods include, but are not limited to: fluorescent in situ hybridization (FISH), direct DNA sequencing, PFGE analysis, Southern blot analysis, single stranded conformation analysis (SSCA), RNAse protection assay, allele specific oligonucleotide (ASO), dot blot analysis, PCR-SSCP, microchip arrays, and WAVE Nucleic Acid Fragment Analysis System as discussed in detail below and in the exemplification.

There are several methods that can be used to detect DNA sequence variation. The process of genotyping may be separated into two general steps: 1) the allele discrimination phase and 2) the analysis phase (for a review of current methodologies see, e.g. Gut, (2001), Hum. Mut. 17:475-492; Kwok, (2001) Annu. Rev. Genomics Hum. Genet, 2:235-58).

Allele Discrimination

Many methods are available for detecting specific alleles at human polymorphic loci. As those skilled in the art will appreciate, the preferred method for detecting a specific polymorphic allele will depend, in part, upon the molecular nature of the polymorphism. For example, the various allelic forms of the polymorphic locus may differ by a single base-pair of the DNA. Such single nucleotide polymorphisms (or SNPs) are major contributors to genetic variation, comprising some 80% of all known polymorphisms, and their density in the human genome is estimated to be on average 1 per 1,000 base pairs. SNPs are most frequently biallelic-occurring in only two different forms (although up to four different forms of an SNP, corresponding to the four different nucleotide bases occurring in DNA, are theoretically possible). Nevertheless, SNPs are mutationally more stable than other polymorphisms, making them suitable for association studies in which linkage disequilibrium between markers and an unknown variant is used to map disease-causing mutations. In addition, because SNPs typically have only two alleles, they can be genotyped by a simple plus/minus assay rather than a length measurement, making them more amenable to automation.

A variety of methods are available for detecting the presence of a particular single nucleotide polymorphic allele in an individual. Advancements in this field have provided accurate, easy, and inexpensive large-scale SNP genotyping. Most recently, for example, several new techniques have been described including dynamic allele-specific hybridization (DASH), microplate array diagonal gel electrophoresis (MADGE), pyrosequencing, oligonucleotide-specific ligation, the TaqMan® system as well as various DNA “chip” technologies such as the Affymetrix SNP chips (Santa Clara, Calif.). These methods require amplification of the target genetic region, typically by PCR. Still other newly developed methods, based on the generation of small signal molecules by invasive cleavage followed by mass spectrometry or immobilized padlock probes and rolling-circle amplification, might eventually eliminate the need for PCR. Several of the methods known in the art for detecting specific single nucleotide polymorphisms are summarized below. The method of the present invention is understood to include all available methods.

The hybridization approach to genotyping exploits the observation that the melting temperature of identical DNA-DNA or DNA-RNA fragments differs from the melting temperature of fragments which differ in sequence. Importantly, under the proper stringency conditions, it is possible to detect even single nucleotide mismatches caused by a single nucleotide polymorphism by this methodology. Methods for stringency manipulation are well known in the art, but it is notable that stringency conditions are affected by the temperature conditions and buffer choice, or by the use of modified oligonucleotides like PNA's (Egholm, et al., (1993) Nature, 365:566-568). Allele specific hybridization forms the basis of, among others, DNA microarray genotyping systems.

Various enzymes, such as DNA polymerases, DNA ligases, or nucleases may additionally be used to better distinguish among different alleles.

One set of methods, amplification refractory mutation system (ARMS) (Newton, et al., (1989) Nucleic Acids Res., 17:2503-2516) and kinetic PCR (Germer et al., (2000) Genome Res., 10:258-266), relies on the observation that oligonucleotides with a mismatched 3′-residue will not function as primers in the PCR under appropriate conditions and therefore the formation or rate of formation of PCR product will be dependant on the allele present in the template. The PCR products obtained with this approach are analyzed for the presence of a SNP by either standard gel electrophoresis (ARMS) or by analyzing the incorporation of fluorescent dye into the PCR products during formation (kinetic PCR).

In one embodiment, SNPs can be identified through the use of the 5′ nuclease assay, also known as TaqMan (Perkin-Elmer). The methodology is known in the art and it requires the addition of a labeled probe, complementary to the internal sequence of a target DNA, to the PCR reaction, where the probe binds to the complementary oligonucleotide carrying the SNP, degrades and fluoresces in the process as the dye and quencher molecule are separated (Holland, et al., (1991); Livak et al., (1995); Kalinina et al., (1997))

In another embodiment, primer extension methodologies of SNP scoring are employed (“minisequencing”) (for a review see, e.g., Syvanen, et al., (1999) Hum. Mut., 13:1-10). In this methodology, an oligonucleotide is annealed adjacent to the polymorphic site in a single stranded PCR amplicon. This step is followed by the addition of DNA polymerase and subsequent enzymatic extension of the primer in the presence of labeled chain terminating dideoxynucleotides. The primer extension method is available in a variety of platforms from multiple companies including, but not limited to, ELISA style microtiter plate formats with calorimetric detection (Orchid BioSciences' SNP-IT™), DNA microarray detection systems (Affymetrix, Amersham Pharmacia's APEX), fluorescent bead-based reaction sorting devices (Luminex), solution phase fluorescence polarization detection systems (LJL Biosystems), mass spectrometry (Sequenom), and automated capillary DNA sequencing (ABI's SNaPshot®).

In another embodiment, oligonucleotide ligation assays (OLA's) are used to distinguish alleles. In this method, oligonucleotides are ligated in the instance where the bases next to the ligation position are fully complementary to the template oligonucleotide (see, e.g., Barany, et al., (1991) P.N.A.S. USA, 88:189-193; Samiotaki et al., (1994) Genomics, 20:238-242; U.S. Pat. No. 4,998,617; Landegren, U. et al. (Science 241:1077-1080 (1988)). The OLA protocol uses two oligonucleotides that are designed to be capable of hybridizing to abutting sequences of a single strand of a target. One of the oligonucleotides is linked to a separation marker, e.g., biotinylated, and the other is detectably labeled. If the precise complementary sequence is found in a target molecule, the oligonucleotides will hybridize such that their termini abut, and create a ligation substrate. Ligation then permits the labeled oligonucleotide to be recovered using avidin, or another biotin ligand. Nickerson, D. A. et al. have described a nucleic acid detection assay that combines attributes of PCR and OLA (Nickerson, D. A. et. al., Proc. Natl. Acad. Sci. (USA) 87:8923-27 (1990)). In this method, PCR is used to achieve the exponential amplification of target DNA, which is then detected using OLA.

Several techniques based on this OLA method have been developed and can be used to detect KCNH2 or KCNQ1 alleles. For example, U.S. Pat. No. 5,593,826 describes an OLA using an oligonucleotide having 3′-amino group and a 5′-phosphorylated oligonucleotide to form a conjugate having a phosphoramidate linkage. In another variation of OLA described in To be et al. (Nucleic Acids Res. 24: 3728 (1996)), OLA combined with PCR permits typing of two alleles in a single microtiter well. By marking each of the allele-specific primers with a unique hapten, i.e., digoxigenin and fluorescein, each OLA reaction can be detected by using hapten specific antibodies that are labeled with different enzyme reporters, alkaline phosphatase or horseradish peroxidase. This system permits the detection of the two alleles using a high throughput format that leads to the production of two different colors.

In another embodiment, the Padlock method may be employed. In this method one nucleotide is circularized by ligation (Nilsson, et al., (1994) Science, 265:2085-2088; Nilsson, et al., (1997) Nat. Genet., 16:252-255; Landegren et al., (1996) Methods, 9:84-90).

In one embodiment of the subject assay, the allele of a KCNH2 or KCNQ1 is identified by alterations in restriction enzyme cleavage patterns with the use of restriction fragment length polymorphism (RFLP) (Myers, Maniatis, and Lerman, Methods Enzymol., 155:501-527 (1987)). For example, sample and control DNA is isolated, amplified (optionally), digested with one or more restriction endonucleases, and fragment length sizes are determined by gel electrophoresis. This method takes advantage of the fact that restriction endonucleases are highly specific for their substrates. In some instances, a SNP will change a recognition site for an restriction enzyme. In such situations, enzymatic digestion will produce a novel set of digested fragments which will be indicative of the presence of the SNP. In one embodiment, this methodology requires electrophoretic size separation techniques, followed by a detection of size differences with a labeled probe DNA fragment on Southern blots. In another embodiment, the DNA may be amplified prior to the digestion in order to conserve the amount of genomic DNA needed for the procedure. Alternatively, novel restriction sites may be introduced to further facilitate the process.

In another embodiment, SNPs are detected with the use of flap endonucleases (FEN) (Harrington and Lieber, (1994) Genes Dev., 8:1344-1355; Mein et al., (2000) Genome Res., 10: 330-343). In this assay, two oligonucleotides, the invader and a signal oligo with a 5′ overhang (the flap), are hybridized to a target oligonucleotide. In the situation where there is a perfect match between the signal oligo and the target, the flap is cleaved off. The cleavage of the 5′ overhang then instigates another reaction which generates a signal (Ryan, et al., (1999) Mol. Diag. 4:135-1440). Importantly, this method does not require the use of PCR. One example of this technology, The Invader™ system, is available from Third Wave Technologies.

In yet another embodiment, any of a variety of sequencing reactions known in the art can be used to directly sequence the allele. Exemplary sequencing reactions include those based on techniques developed by Maxim and Gilbert (Proc. Natl. Acad. Sci. (USA) 74:560 (1977)) or Sanger (Sanger et al., Proc. Nat. Acad. Sci. (USA) 74:5463 (1977)). It is also contemplated that any of a variety of automated sequencing procedures may be utilized when performing the subject assays (see, for example Biotechniques 19:448 (1995)), including sequencing by mass spectrometry (see, for example PCT Publication Nmber WO 94/16101; Cohen et al., Adv. Chromatogr. 36:127-162 (1996); and Griffin et al., Appl. Biochem. Biotechnol. 38:147-159 (1993)). It will be evident to one of skill in the art that, for certain embodiments, the occurrence of only one, two or three of the nucleic acid bases need be determined in the sequencing reaction. For instance, A-track or the like, e.g., where only one nucleic acid is detected, can be carried out.

In another embodiment, single nucleotide polymorphisms are detected with Pyrosequencing—a method that enables continuous reading of short stretches of DNA with the aid of chemiluminiscent reaction that signals each additional base pair extension (Ronaghi et al., (1996) Anal. Biochem., 242:84-89; Ronaghi et al., (1999) Anal. Biochem., 267:65-71).

In one embodiment, the single base polymorphism can be detected by using a specialized exonuclease-resistant nucleotide, as described, e.g., in Mundy, C. R. (U.S. Pat. No. 4,656,127). According to the method, a primer complementary to the allelic sequence immediately 3′ to the polymorphic site is permitted to hybridize to a target molecule obtained from a particular animal or human. If the polymorphic site on the target molecule contains a nucleotide that is complementary to the particular exonuclease-resistant nucleotide derivative present, then that derivative will be incorporated onto the end of the hybridized primer. Such incorporation renders the primer resistant to exonuclease, and thereby permits its detection. Since the identity of the exonuclease-resistant derivative of the sample is known, a finding that the primer has become resistant to exonucleases reveals that the nucleotide present in the polymorphic site of the target molecule was complementary to that of the nucleotide derivative used in the reaction. This method has the advantage that it does not require the determination of large amounts of extraneous sequence data.

In another embodiment of the invention, a solution-based method is used for determining the identity of the nucleotide of a polymorphic site. Cohen, D. et al. (French Patent 2,650,840; PCT Publication No. WO 91/02087). As in the Mundy method of U.S. Pat. No. 4,656,127, a primer is employed that is complementary to allelic sequences immediately 3′ to a polymorphic site. The method determines the identity of the nucleotide of that site using labeled dideoxynucleotide derivatives, which, if complementary to the nucleotide of the polymorphic site will become incorporated onto the terminus of the primer.

An alternative method, known as Genetic Bit Analysis or GBA™ is described by Goelet, P. et al. (PCT Appln. No. 92/15712). The method of Goelet, P. et al. uses mixtures of labeled terminators and a primer that is complementary to the sequence 3′ to a polymorphic site. The labeled terminator that is incorporated is thus determined by, and complementary to, the nucleotide present in the polymorphic site of the target molecule being evaluated. In contrast to the method of Cohen et al. (French Patent 2,650,840; PCT Publication No. WO91/02087) the method of Goelet, P. et al. is preferably a heterogeneous phase assay, in which the primer or the target molecule is immobilized to a solid phase.

Several primer-guided nucleotide incorporation procedures for assaying polymorphic sites in DNA have been described (Komher, J. S. et al., Nucl. Acids. Res. 17:7779-7784 (1989); Sokolov, B. P., Nucl. Acids Res. 18:3671 (1990); Syvanen, A.-C., et al., Genomics 8:684-692 (1990); Kuppuswamy, M. N. et al., Proc. Natl. Acad. Sci. (U.S.A.) 88:1143-1147 (1991); Prezant, T. R. et al., Hum. Mutat 1:159-164 (1992); Ugozzoli, L. et al., GATA 9:107-112 (1992); Nyren, P. et al., Anal. Biochem. 208:171-175 (1993)). These methods differ from GBA™ in that they all rely on the incorporation of labeled deoxynucleotides to discriminate between bases at a polymorphic site. In such a format, since the signal is proportional to the number of deoxynucleotides incorporated, polymorphisms that occur in runs of the same nucleotide can result in signals that are proportional to the length of the run (Syvanen, A.-C., et al., Amer. J. Hum. Genet. 52:46-59 (1993)).

For mutations that produce premature termination of protein translation, the protein truncation test (PTT) offers an efficient diagnostic approach (Roest, et. al., (1993) Hum. Mol. Genet 2:1719-21; van der Luijt, et. al., (1994) Genomics 20:1-4). For PTT, RNA is initially isolated from available tissue and reverse-transcribed, and the segment of interest is amplified by PCR. The products of reverse transcription PCR are then used as a template for nested PCR amplification with a primer that contains an RNA polymerase promoter and a sequence for initiating eukaryotic translation. After amplification of the region of interest, the unique motifs incorporated into the primer permit sequential in vitro transcription and translation of the PCR products. Upon sodium dodecyl sulfate-polyacrylamide gel electrophoresis of translation products, the appearance of truncated polypeptides signals the presence of a mutation that causes premature termination of translation. In a variation of this technique, DNA (as opposed to RNA) is used as a PCR template when the target region of interest is derived from a single exon.

In addition to methods which focus primarily on the detection of one nucleic acid sequence, profiles may also be assessed in such detection schemes. Fingerprint profiles may be generated, for example, by utilizing a differential display procedure, Northern analysis and/or RT-PCR.

Another detection method is allele specific hybridization using probes overlapping a region of at least one allele of a KCNH2 or KCNQ1 haplotype and having about 5, 10, 20, 25, or 30 nucleotides around the mutation or polymorphic region. In a preferred embodiment of the invention, several probes capable of hybridizing specifically to other allelic variants involved in a cardiovascular disorder are attached to a solid phase support, e.g., a “chip” (which can hold up to about 250,000 oligonucleotides). Oligonucleotides can be bound to a solid support by a variety of processes, including lithography. Mutation detection analysis using these chips comprising oligonucleotides, also termed “DNA probe arrays” is described, e.g., in Cronin et al., Human Mutation 7:244 (1996). In one embodiment, a chip comprises all the allelic variants of at least one polymorphic region of a gene. The solid phase support is then contacted with a test nucleic acid and hybridization to the specific probes is detected. Accordingly, the identity of numerous allelic variants of one or more genes can be identified in a simple hybridization experiment.

In a further embodiment, protection from cleavage agents (such as a nuclease, hydroxylamine or osmium tetroxide and with piperidine) can be used to detect mismatched bases in RNA/RNA or RNA/DNA or DNA/DNA heteroduplexes (Myers, et al., Science 230:1242 (1985)). In general, the art technique of “mismatch cleavage” starts by providing heteroduplexes formed by hybridizing (labeled) RNA or DNA containing the wild-type allele with the sample. The double-stranded duplexes are treated with an agent which cleaves single-stranded regions of the duplex such as which will exist due to base pair mismatches between the control and sample strands. For instance, RNA/DNA duplexes can be treated with RNase and DNA/DNA hybrids treated with S1 nuclease to enzymatically digest the mismatched regions. In other embodiments, either DNA/DNA or RNA/DNA duplexes can be treated with hydroxylamine or osmium tetroxide and with piperidine in order to digest mismatched regions. After digestion of the mismatched regions, the resulting material is then separated by size on denaturing polyacrylamide gels to determine the site of mutation. See, for example, Cotton et al., Proc. Natl. Acad. Sci. (USA) 85:4397 (1988); and Saleeba et al., Methods Enzymol. 217:286-295 (1992). In a preferred embodiment, the control DNA or RNA can be labeled for detection.

In still another embodiment, the mismatch cleavage reaction employs one or more proteins that recognize mismatched base pairs in double-stranded DNA (so called “DNA mismatch repair” enzymes). For example, the mutY enzyme of E. coli cleaves A at G/A mismatches and the thymidine DNA glycosylase from HeLa cells cleaves T at GfT mismatches (Hsu et al., Carcinogenesis 15:1657-1662 (1994)). According to an exemplary embodiment, a probe based on an allele of a haplotype is hybridized to a cDNA or other DNA product from a test cell(s). The duplex is treated with a DNA mismatch repair enzyme, and the cleavage products, if any, can be detected from electrophoresis protocols or the like. See, for example, U.S. Pat. No. 5,459,039.

Examples of other techniques for detecting alleles include, but are not limited to, selective oligonucleotide hybridization, selective amplification, or selective primer extension. For example, oligonucleotide primers may be prepared in which the known mutation or nucleotide difference (e.g., in allelic variants) is placed centrally and then hybridized to target DNA under conditions which permit hybridization only if a perfect match is found (Saiki et al., Nature 324:163 (1986)); Saiki et al., Proc. Natl. Acad. Sci. (USA) 86:6230 (1989)). Such allele specific oligonucleotide hybridization techniques may be used to test one mutation or polymorphic region per reaction when oligonucleotides are hybridized to PCR amplified target DNA or a number of different mutations or polymorphic regions when the oligonucleotides are attached to the hybridizing membrane and hybridized with labeled target DNA.

Alternatively, allele specific amplification technology which depends on selective PCR amplification may be used in conjunction with the instant invention. Oligonucleotides used as primers for specific amplification may carry the mutation or polymorphic region of interest in the center of the molecule (so that amplification depends on differential hybridization) (Gibbs et al., Nucleic Acids Res. 17:2437-2448 (1989)) or at the extreme 3′ end of one primer where, under appropriate conditions, mismatch can prevent, or reduce polymerase extension (Prossner, Tibtech 11:238 (1993). In addition it may be desirable to introduce a novel restriction site in the region of the mutation to create cleavage-based detection (Gasparini et al., Mol. Cell Probes 6:1 (1992)). It is anticipated that in certain embodiments amplification may also be performed using Taq ligase for amplification (Barany, Proc. Natl. Acad. Sci. (USA) 88:189 (1991)). In such cases, ligation will occur only if there is a perfect match at the 3′ end of the 5′ sequence making it possible to detect the presence of a known mutation at a specific site by looking for the presence or absence of amplification.

SNP Analysis Methods

Multiple analysis methodologies are available for the analysis of potential allelic variants. Such methods include, but are not limited to, gels, oligonucleotide microarrays, coded spheres, mass spectrometers, and microtiter plate fluorescent readers with integrated thermocyclers.

In one embodiment, gel analysis is used in combination with DNA sequencing. In another embodiment, MASDA, a hybridization based genotyping method that includes gel analysis may be used (Shuber, et al., (1997) Hum. Mol. Genet. 6:337-347). Other utilizations of the gel analysis approach include, but are not limited to: primer extension methods combined with slab gel fluorescent analysis (Pastinen, et al., (1996) Clin. Chem. 42:1391-1397), oligonucleotide ligation assay combined with gel analysis (Grossman, et al., (1994) Nucleic Acids Res. 22(21):4527-34; Day et al., (1995) Genomics, 29:152-62), microtiter array diagonal gel electrophoresis (MADGE) (Day and Humphries, (1994) Anal. Biochem. 222(2):389-95); restriction digest allele detection combined with gel based analysis (RFLP) (Parsons and Heflich, (1997) Mutat. Res. 387(2):97-121); primer extension methods combined with 96-capillary electrophoresis sequencers (e.g., SNapShot from Applied Biosystems); the hybridization method combined with MASDA analysis (Shuber, et al., (1997) Hum. Mol. Gen. 6:337-347); the ligation method combined with Padlock analysis (Nilsson, et al., (1994); Landegren, et al., (1996)).

In yet another embodiment, alterations in electrophoretic mobility may be used to identify a KCNH2 or a KCNQ1 allele. For example, single strand conformation polymorphism (SSCP) may be used to detect differences in electrophoretic mobility between mutant and wild type nucleic acids (Orita et al., Proc Natl. Acad. Sci (USA) 86:2766 (1989), see also Cotton, Mutat. Res. 285:125-144 (1993); and Hayashi, Genet. Anal. Tech. Appl. 9:73-79 (1992)). Single-stranded DNA fragments of sample and control KCNH2 or KCNQ1 alleles are denatured and allowed to renature. The secondary structure of the single-stranded nucleic acids varies according to sequence resulting in an alteration of the electrophoretic mobility that enables the detection of even a single base change. The DNA fragments may be labeled or detected with labeled probes. The sensitivity of the assay may be enhanced by using RNA (rather than DNA), in which the secondary structure is more sensitive to a change in sequence. In a preferred embodiment, the subject method utilizes heteroduplex analysis to separate double stranded heteroduplex molecules on the basis of changes in electrophoretic mobility (Keen et al., Trends Genet 7:5 (1991)).

In yet another embodiment, the movement of alleles in polyacrylamide gels containing a gradient of denaturant is assayed using denaturing gradient gel electrophoresis (DGGE) (Myers et al., Nature 313:495 (1985)). When DGGE is used as the method of analysis, DNA may be modified to prevent complete denaturation, for example, by adding a GC clamp of approximately 40 bp of high-melting GC-rich DNA by PCR. In a further embodiment, a temperature gradient is used in place of a denaturing agent gradient to identify differences in the mobility of control and sample DNA (Rosenbaum and Reissner, Biophys. Chem. 265:12753 (1987)).

Allele detection technologies may also be combined with fluorescene energy transfer as a means of analysis (for a review see, Gut, I., (2001) Hum. Mut. 17:475-492). For example, this methodology may be used in combination oligonucleotide ligation (Chen, X., et al., (1998) Genome Res., 8: 549-556), or in combination with a primer extension procedure (Chen, X., et al., (1997) Nucleic Acid Res. 25: 347-353; Chen, X., et al., (1999) Genome Res. 9: 492-498).

Allele detection technologies may also be combined with microarrays or mass spectroscopy as a means of analysis (for a review see, Gut, I., (2001) Hum. Mut. 17:475-492).

Genotyping Kits

Other embodiments of the invention are directed to kits for detecting a predisposition for developing a drug-induced arrhythmia, either as a result of the administration of anti-arrhythmic or non-cardiac medication. These kits may contain one or more oligonucleotides, including 5′ and 3′ oligonucleotides that hybridize 5′ and 3′ to at least one allele of either KCNH2 or KCNQ1. PCR amplification oligonucleotides should hybridize between 25 and 2500 base pairs apart, preferably between about 100 and about 500 bases apart, in order to produce a PCR product of convenient size for subsequent analysis.

The oligonucleotides present in one embodiment of a kit according to the present invention may be used for amplification of the region of interest or for direct allele specific oligonucleotide (ASO) hybridization to the markers in question. Thus, the oligonucleotides may either flank the marker of interest (as required for PCR amplification) or directly overlap the marker (as in ASO hybridization).

The design of oligonucleotides for use in the amplification and detection of either KCNH2 or KCNQ1 polymorphic alleles by the method of the invention is facilitated by the availability of both updated sequence information from human chromosome 11 (specific location 11 p15.5), which contains the human KCNH2 locus, or chromosome 7 (specific location 7q35-q36) which contains the human KCNQ1 locus, and updated human polymorphism information available for this locus. Suitable primers for the detection of a human polymorphism in these genes can be readily designed using this sequence information and standard techniques known in the art for the design and optimization of primers sequences. Optimal design of such primer sequences can be achieved, for example, by the use of commercially available primer selection programs such as Primer2.1, Primer3 or GeneFisher®.

For use in a kit, oligonucleotides may be any of a variety of natural and/or synthetic compositions such as synthetic oligonucleotides, restriction fragments, cDNAs, synthetic peptide nucleic acids (PNAs), and the like. The assay kit and method may also employ labeled oligonucleotides to allow ease of identification in the assays. Examples of labels which may be employed include radio-labels, enzymes, fluorescent compounds, streptavidin, avidin, biotin, magnetic moieties, metal binding moieties, antigen or antibody moieties, and the like.

The kit may, optionally, also include DNA sampling means. DNA sampling means are well known to one of skill in the art. Reagents for DNA sampling include DNA purification reagents such as Nucleon™ kits, lysis buffers, proteinase solutions and the like; PCR reagents, such as 10× reaction buffers, thermostable polymerase, dNTPs, and the like; and allele detection means such as the Hinfl restriction enzyme, allele specific oligonucleotides, and degenerate oligonucleotide primers for nested PCR from dried blood.

The practice of the present invention will employ, unless otherwise indicated, conventional techniques of cell biology, cell culture, molecular biology, transgenic biology, microbiology, recombinant DNA, and immunology, which are within the skill of the art. Such techniques are described in the literature. See, for example, Molecular Cloning A Laboratory Manual, 2nd Ed., ed. by Sambrook, Fritsch and Maniatis (Cold Spring Harbor Laboratory Press: 1989); DNA Cloning, Volumes 1 and II (D. N. Glover ed., 1985); Oligonucleotide Synthesis (M. J. Gait ed., 1984); Mullis et al. U.S. Pat. No. 4,683,195; Nucleic Acid Hybridization (B. D. Hames & S. J. Higgins eds. 1984); Transcription And Translation (B. D. Hames & S. J. Higgins eds. 1984); Culture Of Animal Cells (R. I. Freshney, Alan R. Liss, Inc., 1987); Immobilized Cells And Enzymes (IRL Press, 1986); B. Perbal, A Practical Guide To Molecular Cloning (1984); the treatise, Methods In Enzymology (Academic Press, Inc., N.Y.); Gene Transfer Vectors For Mammalian Cells (J. H. Miller and M. P. Calos eds., 1987, Cold Spring Harbor Laboratory); Methods In Enzymology, Vols. 154 and 155 (Wu et al. eds.), Immunochemical Methods In Cell And Molecular Biology (Mayer and Walker, eds., Academic Press, London, 1987); Handbook Of Experimental Immunology, Volumes I-IV (D. M. Weir and C. C. Blackwell, eds., 1986); Manipulating the Mouse Embryo, (Cold Spring Harbor Laboratory Press, Cold Spring Harbor, N.Y., 1986).

Exemplification

Molecular Genetics of Arrhvthmias—Cardiac Potassium Channels

The susceptibility to arrhythmias is at least partially determined by aberrations in various ion channel genes. Arrhythmias may be further categorized into inherited arrhythmias, idiopathic arrhythmias, and drug-induced arrhythmias.

To date, six independent genes, encoding individual ion channel subunits, have been identified as playing a role in the pathogenesis of inherited arrhythmias, including long QT syndrome (LQTS): KCNQL (LQT1), KCNH2 (h-erg or LQT2), SCN5A (LQT3), KCNE1 (LQT5), KCNE2 (MiRP1 or LQT6), and RyR2 (Curran, et al., (1995) Cell, 80: 795-803; Wang, et al., (1995) Cell 80:805-811; Wang, et al., (1995) Hum. Mol. Genet. 4:1603-1607; Wang, et al., (1996) Nat. Genet. 12:17-23; Splawski, et al., (1997) N. Engl. J. Med., 336:1562-1567; Splawski, et al., (1997) Nat. Genet., 17:338-340; Chen et al., (1998) Nature, 392:293-296; Abbott et al., (1999) Cell, 97:175-187; Priori, et al., (2000) Circulation, 102:r49-r53). The KCNQ1, KCNE1, KCNE2 and KCNH2 nucleic acids encode potassium channel subunits, while the SCN5A nucleic acid encodes a sodium channel subunit, and RyR2 encodes for a cardiac ryanodine receptor.

Functional ion channels are multimers of individual subunits which can combine in either homomeric or heteromeric combinations. In the instance of cardiac potassium channels, KCNH2 subunits co-assemble into homo-multimeric potassium channels, while KCNE1 and KCNQ1 (KVLQT1) channel subunits co-assemble to form hetero-multimeric potassium channel proteins.

Recent advances in molecular genetics have helped to elucidate the genetic basis of most cases of inherited long QT syndrome (LQTS) which is characterized by recurrent syncope (often during exercise or emotional stress), prolongation of the QT interval and T and U wave abnormalities on the ECG. This in turn has lead to an emerging association of these genes relation to drug-induced, or acquired, prolongation of QT interval. One cause of syncope is TdP which may progress to ventricular fibrillation and death. The mechanism of the development of TdP is similar in both acquired and inherited TdP: a failure of normal complete membrane repolarisation by the blockade of outward repolarising K⁺ channels or the activation of inward depolarising Na⁺ channels. Failure of normal depolarization becomes a trigger for after depolarizations, which can result in the prolongation of the QT interval and the initiation of ventricular arrhythmias. This suggests that there may be links between acquired and inherited forms of the long QT syndrome.

The majority of LQTS subjects appear to harbor mutations in either of two cardiac potassium channel genes, KCNH2 and KCNQ1 (see Curran, M. E., et al., Cell 80:795-803 (1995) and Wang, Q., et al., Nature Genet 12:17-23 (1996)), while additional cases are caused by mutations in genes encoding potassium channel regulatory subunits KCNE1 and KCNE2 (see Splawski, I., et al., Nature Genet. 17:338-340 (1997); Abbott, G. W., et al., Cell 97:175-187 (1999)), a cardiac voltage-dependent sodium channel alpha subunit SCN5A (see Wang, Q., et al., Cell 80:805-811 (1995)), and other unidentified gene products (see Schott, J. J., et al., 57:1114-1122 (1995)).

Many of LQTS belong to an autosomal dominant form of the disorder, the Romano-Ward syndrome. A rare autosomal recessive variant, the Jervell-Lange-Nielsen syndrome is associated with deafness. The Romano-Ward syndrome is genetically heterogeneous with phenotypic differences in severity and outcome. Mutations in four different genes (LQT1, LQT2, LQT3 and LQT5), located on chromosomes 11, 7, 3 and 21 respectively are responsible for more than 90% of LQTS. A fifth locus on chromosome 4 has been associated with the disorder in a French pedigree and other as yet unidentified genes (i.e., excluded by linkage analysis to previously identified genes) account for the remainder.

LQT1: approximately 50% of the incidence of LQTS, involves a mutation in a newly cloned gene KCNQ1 which encodes a 581 amino acid protein similar to voltage activated potassium channels. In combination with KCNE1 it appears to constitute a delayed rectifying potassium current (IK_{S}) responsible for phase 3 repolarisation in the heart. To date approximately 35 mutations have been identified for this gene.

LQT2: mutations in a second potassium channel gene (KCNH2) have been identified in LQTS and linked to chromosome 7. KCNH2 is responsible for the rapidly activating delayed rectifier potassium current (IK_{R}), which is also involved in phase 3 repolarisation. Class III anti-arrhythmic drugs such as dofetilide block the KCNH2 channel. At least 17 mutations have been identified for this gene.

LQT3, is linked to the gene for a sodium channel (SCN5A) located on chromosome 3. Three mutations have so far been identified and these result in failure to rapidly inactivate the channel resulting in prolonged bursts of activity.

LQT5: KCNE1 located on chromosome 21 codes for a potassium channel protein which interacts functionally with the protein encoded by KCNQ1 to form an ion current with the properties of I_{KS}. Compound heterozygous mutations, Thr7lle and Asp76Asn, were found in a family with the Jervell-Lange-Nielsen syndrome. Additionally, researchers have determined that the Asp76Asn variation and another mutation, Ser74Leu, in patients with the long QT syndrome reduce I_{KS} by shifting the voltage dependence of activation and accelerating channel deactivation.

Implications for Drug-Induced TdP

Far less is known about the etiology of drug-induced arrhythmias, although evidence in the literature as well as that disclosed herein indicate that ion channel genes are similarly the molecular culprits of the disorder.

K⁺ or Na⁺ ion channel dysfunction is related to the characterized mutation. In KCNH2 for example missense mutations generally result in a more severe reduction in channel function than do deletions. However different missense mutations may also have different effects: Y611H and V822M result in abnormal channel processing so that the protein is retained within the endoplasmic reticulum; 1593G and G628S are processed normally but produce non-functioning channels; whilst T474I produces altered channel gating. Thus individuals could carry subclinical mutations which are manifest only upon challenge with drugs which block K⁺ channels or prolong the action potential. In KCNQ1, mutations, which lead to substitutions in the carboxy terminal end of the channel, appear to result in a less severe phenotype.

Until recently it was believed that all mutations giving rise to the long QT syndrome exerted a dominant negative effect, i.e., heterozygotes were affected and the inheritance was autosomal dominant. Recently a functional mutation was discovered in a family with LQT1, which showed autosomal recessive inheritance. All heterozygotes had normal QT intervals. The functional mutation resulted in a milder impairment of channel activity (a reduction in total current, a hyperpolarising shift in activation and a faster activation rate). This suggests that mild mutations in LQTS genes may exist in the general population and possibly at a higher prevalence than genes, which inevitably result in clinical consequences.

Recently a missense mutation in the KCNH2 gene in a 59 year-old female who developed aLQTS after treatment with quinidine has been identified. Additionally, a mutation in the KCNQ1 gene in a 74 year old woman who presented with TdP whilst receiving treatment with cisapride as well as a 30 year old woman with inherited long QTS developed TdP after taking astemizole and erythromycin.

The invention now being generally described will be more readily understood by reference to the following examples which are included merely for purposes of illustration of certain aspects and embodiments of the present invention, and are not intended to limit the invention in any way.

### EXAMPLE 1

### DNA Isolation and Amplification

Genomic DNA samples were obtained from the blood samples of patients enrolled in the DIAMOND study (Danish Investigations of Arrhythmia and Mortality on Dofetilide) or a clinical protocol similar in nature to the DIAMOND Study. These studies focused on the effects of Dofetilide on two populations of patients, one with congestive heart failure and one with recent myocardial infarction associated with left ventricular dysfunction (ejection fraction <35%) (Moller, M., (1996) Lancet, 348(9041):1597-8; Moller, M. et al., (1997) Congest Heart Fail., 7(3):146-150). DNA samples were obtained from 105 patients in total. Of the 105 patients, 7 developed torsade de pointes (TdP) upon treatment with Dofetilide while the remaining 98 patients responded to treatment without developing TdP as a side effect.

### EXAMPLE 2

### Screening Methods

Isolated DNA was amplified using PCR methodologies described in Neyroud, N., et al., (1999) Circ. Res. 84:290-297. The primers were constructed on the basis of the flanking intron sequences and were used to amplify each exon.

Mutational screening was performed either with the use of the automated WAVE Nucleic Acid Fragment Analysis System (Transgenomic, Kuklin, A., et al., (1997) Genet. Test., 1(3):201-6; Kuklin, A., et al., (1999) Mol. Cell. Probes, 13(3):239-42) or direct sequencing methods. For KCNQ1, mutation screening was carried out with WAVE and the presence of candidate polymorphisms was independently verified using conventional di-deoxy sequencing techniques (see, e.g., Sanger et al., (1977) Proc. Nat. Acad. Sci (USA) 74:5463). For KCNH2, mutation screening was carried out with conventional di-deoxy sequencing techniques. After the confirmation of mutations with sequencing, genotyping by the fluorescence polarization method (FP), (Chen, X., et al., (1999) Genome Research, 9:492-498), was performed on 96 controls (192 chromosomes). The WAVE Nucleic Acid Fragment Analysis System couples heteroduplex analysis (HA) with Denaturing High Performance Liquid Chromatography (DHPLC) and can be used to detect variants in PCR products containing both allelic forms of a polymorphism (either heterozygotes or a 1:1 mix of both alleles) via heteroduplex separation. The FP methodology relies on the observation that the degree of fluorescence polarization emitted by a molecule will be proportional to the molecular weight of that molecule and therefore the degree of polarization will change in instances where the molecule of interest contains a polymorphism (for a review see, Kwok, P.Y., (2002) Human Mut., 19:315-323).

### EXAMPLE 3

### Screening Cardiac Ion Channels for Polymorphisms Underlying Drug-induced Arrhythmia

Candidate genes for LQTS were screened for the presence of novel polymorphisms. The candidate LQTS genes screened in this study, KCNQ1, KCNH2, KCNE1, KCNE2, SCN5A, all encode cardiac ion channels. A brief summary of the ion channel genes, their locus and chromosomal location, the encoded protein, and the protein function is provided below:

| | CHROMOSOMAL | | | |
|---|---|---|---|---|
| LOCUS | LOCATION | GENE | PRODUCT | FUNCTION |
| LQT1 | 11p15.5 | KCNQ1 | K⁺ Channel | Structural |
| | | | | alpha subunit |
| | | | | I_{Ks} |
| LQT2 | 7q35-36 | KCNH2 | K⁺ Channel | Structural |
| | | | | alpha subunit |
| | | | | I_{Kr} |
| LQT3 | 3p21-24 | SCN5A | Na⁺ Channel | Function |
| | | | | Sodium |
| | | | | Channel |
| LQT4 | 4q25-27 | ? | ? | ? |
| LQT5 | 21q22.1 | KCNE1 | KCNE1 | Reglatory beta |
| | | | | subunit I_{Ks} |
| LQT6 | 21q22.1 | KCNE2 | KCNE2 | Reglatory beta |
| | | | | subunit I_{Kr} |

The presence of a novel polymorphism in patients who developed TdP in response to Dofetilide but not in control Dofetilide treated patients was interpreted as indicating that these novel mutations play a role in the molecular genetics of drug-induced arrhythmia. The results of the screen are show in the table below:

Subjects

| Ion | | | | | | | | |
|---|---|---|---|---|---|---|---|---|
| Channel | #1 | #2 | #3 | #4 | #5 | #6 | #7 | Control |
| KCNQ1 | | | present | | | | | 0/196 |
| K218E | | | | | | | | |
| KCNH2 | | | present | present | | | | 5/196 |
| R1047L | | | | | | | | |
| KCNE1 | | | present | present | | | present | ND |
| G38S | | | | | | | | |
| SCN5A | | | present | present | | | present | ND |
| C/A Intron | | | | | | | | |

Of the identified mutations, only the amino acid substitutions in the KCNQ1 and KCNH2 genes were determined to be novel changes with putative functional significance. Additionally, 98 controls (196 chromosomes) were tested for the presence of the KCNQ1 or KCNH2 mutations. The G38S variant in KCNE1 was determined to be a common variant that was previously identified, while the C/A intron found in SCN5A was novel but with unknown significance.

### EXAMPLE 4

### Identification of a KCNQ1 Polymorphism in Patients with Dofetilide Induced TdP

variations were identified the seven patients who developed TdP following treatment with Dofetilide using the WAVE SNP detection methodology including A652G in one patient (#3), G1638A in two patients (#3 and #5), C1986T in two patients (#2 and #4), T2295C in one patient (#4), A2421 G in four patients (#1, #2, #4 and #6) and C2442T in one patient (#6). Except for A652G, the none of the other variations were pursued further as these did not appear to have functional significance (e.g., silent mutation, intronic, etc.).

For KCNQ1 mutation screening was carried out with WAVE and the identified variants were confirmed with sequencing. After the confirmation of mutations, genotyping by the fluorescence polarization method (FP), (Chen, X., et al., (1999) Genome Research, 9:492-498), was performed on 98 controls (196 chromosomes). The FP reverse primer was: GCCGACGTGGCAAACACCTGCCCCT (SEQ ID NO:9).

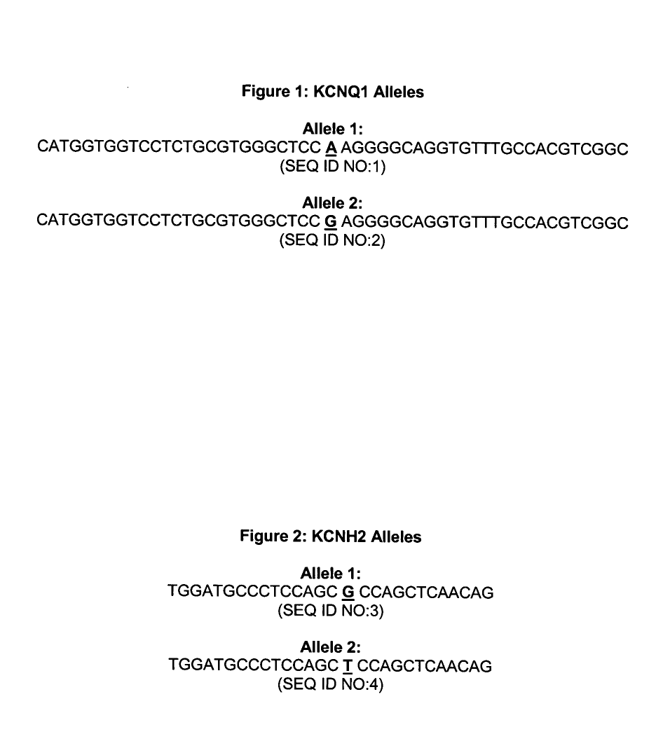

The KCNQ1 polymorphism is an A to G transition at base number 652 of the KCNQ1 gene (A652G). The polymorphism is flanked by sequences: CATGGTGGTCCTCTGCGTGGGCTCC A/G GGGGCAGGTGTTTGCCACGTCGGC (SEQ ID: 10), as shown in FIG. 1. The single base pair transition changes the triplet code from AAG to GAG and results in a lysine (K) to glutamic acid (E) amino acid substitution at position 218 in the amino acid sequence (K218E).

In the seven patients who developed TdP following treatment with Dofetilide, the A652G SNP corresponding to a K218E amino acid substitution, was detected with a frequency of 0.07/0.93 (1 mutation/14 chromosomes). In the 98 control patients who did not develop TdP following treatment with Dofetilide, the A652G SNP corresponding to a K218E amino acid substitution, was detected with a frequency of 0.0.

### EXAMPLE 5

### Identification of a KCNH2 Polymorphism in Patients with Dofetilide Induced TdP

Oligonucleotide primer pairs used to amplify the KCNH2 coding sequence were: (1) forward primer: TGTAAAACGACGGCCAGTTACCCCGCTCACCCAGCTCTGCTC (SEQ ID NO: 11) reverse primer: CAGGAAACAGCTATGACCCCCCCACCCCACTTGCATTCCTTC (SEQ ID NO: 12). The KCNH2 polymorphism was identified by direct sequencing. Additionally, genotyping by the fluorescence polarization method (FP), (Chen, X., et al., (1999) Genome Research, 9:492-498), was performed on 98 controls (196 chromosomes).The FP forward primer was: GAGAGCAGGCTGGATGCCCTCCAGC (SEQ ID NO: 13). Briefly, the protocol required 2.0 μl 5× buffer, 0.05 μl 25 μM G/T mix, 0.1 μl 100 μM forward FP primer, 0.025 μl Thermosequenase, 7.825 μl water.

The KCNH2 polymorphism is a G to T transition at base number 3140 of the KCNH2 gene (G3140T). The exon 13 polymorphism is flanked by sequences: TGGATGCCCTCCAGC G/T CCAGCTCAACAG (SEQ ID NO: 14), as shown in FIG. 2. The single base pair transition changes the triplet code from CGC to CTC and results in a arginine (R) to leucine (L) amino acid substitution at position 1047 in the amino acid sequence (R1047L).

In the seven patients who developed TdP following treatment with Dofetilide, the G3140T SNP corresponding to a R1047L amino acid substitution, was detected with a frequency of 0.21/0.79 (3 mutations/14 chromosomes) (as determined by sequencing (5 wildtype, 1 heterozygote, 1 homozygous mutant). In the 98 control patients who did not develop TdP following treatment with Dofetilide, the G3140T SNP corresponding to a R1047L amino acid substitution, was detected with a frequency of 0.02/0.98 as determined by FP (94 wiltype, 4 heterozygotes).

### Equivalents

Those skilled in the art will recognize, or be able to ascertain using no more than routine experimentation, many equivalents to the specific embodiments of the invention described herein. Such equivalents are intended to be encompassed by the following claims.

Unless otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in this specification and attached claims are approximations that may vary depending upon the desired properties sought to be obtained by the present invention.

All publications and patents mentioned herein, including those items listed below, are hereby incorporated by reference in their entirety as if each individual publication or patent was specifically and individually indicated to be incorporated by reference. In case of conflict, the present application, including any definitions herein, will control. Also incorporated by reference in their entirety are any polynucleotide and polypeptide sequences which reference an accession number correlating to an entry in a public database, such as those maintained by The Institute for Genomic Research (TIGR) and/or the National Center for Biotechnology Information (NCBI). Also incorporated by reference are the following: U.S. Pat. No. 6,458,542; U.S. Pat. No. 6,355,434; U.S. Pat. No. 6,291,175; U.S. Pat. No. 5,834,200; U.S. Pat. No. 6,280,941; U.S. Pat. No. 6,297,014; 20020032319; U.S. Pat. No. 6,475,736; U.S. Pat. No. 6,440,707; U.S. Pat. No. 6,428,964; U.S. Pat. No. 6,262,250; U.S. Pat. No. 6,235,474; U.S. Pat. No. 5,834,200; U.S. Pat. No. 5,712,098; U.S. Pat. No. 5,529,900; U.S. Pat. No. 6,432,644; U.S. Pat. No. 6,207,383; U.S. Pat. No. 6,342,357; U.S. Pat. No. 5,599,673; 20010034024; U.S. Pat. No. 6,475,736. Also incorporated by reference are sequences referred to by their GenBank Accession Numbers throughout the application, as well as, the following sequences: AJ006345, AB009069, AB00905S01, AB00905S02, AB00905S03, AB00905S04, AB00905S05, AB00905S06, AB00905S07, AB00905S08, AB00905S09, AB00905S10, AB00905S11, AB00905S12, AB00905S13, AB00905S14, AB00905S15, AB044806, AF032897, NM000218.

1. A method for screening a human subject for susceptibility to a drug-induced cardiac arrhythmia, said method comprising detecting a R1047L polymorphism in a KCNH2 nucleic acid obtained from said subject, the presence of said R1047L polymorphism indicating an increased susceptibility of said subject to said drug-induced arrhythmia.

2. The method of claim 1, wherein said drug-induced arrhythmia is associated with a long QT interval.

3. The method of claim 1, wherein said drug-induced arrhythmia is Torsade de Pointes.

4. The method of claim 1, wherein said drug is a class III anti-arrhythmic.

5. The method of claim 4, wherein said drug is Dofetilide.

6. A method for screening a human subject for susceptibility to a drug-induced cardiac arrhythmia, said method comprising detecting a K218E polymorphism in a KCNQ1 nucleic acid obtained from said subject, the presence of said K218E polymorphism indicating an increased susceptibility of said subject to said drug-induced arrhythmia.

7. The method of claim 6, wherein said drug-induced arrhythmia is associated with a long QT interval.

8. The method of claim 6, wherein said drug-induced arrhythmia is Torsade de Pointes.

9. The method of claim 6, wherein said drug is a class III anti-arrhythmic.

10. The method of claim 9, wherein said drug is Dofetilide.

11. The method of claim 1, wherein said R1047L polymorphism comprises a G to T transition at nucleotide 16 of a DNA that corresponds to SEQ ID NO:3.

12. The method of claim 2, wherein said K218E polymorphism comprises an A to G transition at nucleotide 26 of a DNA that corresponds to SEQ ID NO:1.

13. An isolated nucleic acid comprising at least 11 consecutive nucleotides of SEQ ID NO: 2 wherein position 26 is a G, corresponding to a K to E amino acid substitution.

14. A nucleic acid probe which hybridizes to SEQ ID NO:2 under conditions at which it will not hybridize to a nucleic acid of SEQ ID NO:1.

15. An isolated nucleic acid comprising at least 11 consecutive nucleotides of SEQ ID NO:4 wherein position 16 is a T, corresponding to a R to L amino acid substitution.

16. A nucleic acid probe which hybridizes to SEQ ID NO:4 under conditions at which it will not hybridize to a nucleic acid of SEQ ID NO:3.
